SEQUENCE LISTING (3 sequences)

SEQ ID NO: 1 - protein, 157 aa, Homo sapiens [sequence omitted]

SEQ ID NO: 2 - protein, 13 aa, Synthetic construct
LGEYGFQNALIVR

SEQ ID NO: 3 - protein, 112 aa, Homo sapiens
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

(19) United States
(12) Patent Application Publication
(10) Pub. No.: US 2025/0076308 A1
(43) Pub. Date: Mar. 6, 2025

(54) COMPOSITIONS AND METHODS FOR PLASMA-FREE TRIFLUOROMETHYL AND HYDROXYL RADICAL LABELING

(71) Applicant: Immuto Scientific, Madison, WI (US)

(72) Inventors: Daniel Benjamin, Madison, WI (US); Faraz Choudhury, Fitchburg, WI (US); Jonathan Schmitz, Madison, WI (US); James Dowell, Middleton, WI (US); Alexis Lawton, Madison, WI (US); Samantha Knott, Madison, WI (US); Patric Sadecki, Madison, WI (US)

(21) Appl. No.: 18/826,449

(22) Filed: Sep. 6, 2024

Related U.S. Application Data
(60) Provisional application No. 63/536,870, filed on Sep. 6, 2023.

(51) Int. Cl. G01N 33/68

(52) U.S. Cl. CPC G01N 33/6848; G01N 2458/15

(57) ABSTRACT

Provided herein are materials and methods for plasma-free labeling of biological molecules with trifluoromethyl and hydroxyl radicals generated using a peroxide and a radical precursor such as sodium triflinate. The methods provided herein find use in labeling of molecules, such as cell-membrane proteins, in live, intact cells.

### PRIORITY STATEMENT

This application claims priority to U.S. Provisional Application No. 63/536,870, filed Sep. 6, 2023, the entire contents of which are incorporated herein by reference for all purposes.

### SEQUENCE LISTING

The text of the computer readable sequence listing filed herewith, titled “IMMUT_42354_202_SequenceListing.xml”, created Sep. 6, 2024, having a file size of 3,953 bytes, is hereby incorporated by reference in its entirety.

### TECHNICAL FIELD

Provided herein are materials and methods for plasma-free labeling of biological molecules with trifluoromethyl and hydroxyl radicals generated using a peroxide such as tert-butyl hydroperoxide (TBHP) and a radical precursor such as sodium triflinate.

### BACKGROUND

Chemical labeling of proteins coupled to mass spectrometry (MS), such as hydrogen-deuterium exchange (HDX) and radical footprinting, are a family of techniques which have been fundamental for the understanding of protein higher order structure (HOS) and the field of structural biology. The large sample requirements, protein size limitations and time for crystallization of high-resolution techniques, such as cryogenic electron microscopy (cryo-EM) and x-ray crystallography, often make highly dynamic protein samples less amenable to study. Although footprinting cannot provide the atomic resolution of crystallography, it is considerably higher throughput. Additionally, the resolution from a footprinting experiment approaches single amino acid level and rivals throughput of techniques such as peptide scanning or epitope binning, which are often limited to the peptide level.

The most popular and frequently applied protein footprinting technique is Hydrogen Deuterium Exchange (HDX), which is based on the principle of protein backbone amide proton exchange and labeling with deuterium. Despite its popularity, HDX-MS is hindered by the inherent shortcoming of back exchange and the reversibility of deuterium labeling, which can result in a significant loss of signal. Additionally, the resulting data is generally limited to the peptide level resolution.

Radical based protein footprinting provides an attractive alternative to HDX, as the chemical moieties are covalently and irreversibly bound. Chemical labeling utilizing radical species can be introduced in several ways for protein footprinting and can yield both peptide and residue level information. The leading footprinting approach is Hydroxyl Radical Protein Footprinting (HRF), where hydroxyl (·OH) radicals are generated in solution. Although HRF is a powerful technique for analysis of protein structure, motion, and interactions, it can be limited by resolution. Due to kinetics and reactivity rates, OH radicals typically only routinely label 6 out of the 20 amino acids: methionine (M), cystine (C), tryptophan (W), phenylalanine (F), histidine (H) and tyrosine (Y). Considering these residues may only cover-5-30% of the protein sequence, the resolution of the data from hydroxyl radical footprinting (HRF) experiments is limited by the ability to acquire information on regions containing these six residues. Alternatively, covalent, chemical labeling approaches, such as diethyl pyrocarbonate (DEPC) have been introduced to label ˜30% of the protein (histidine (H), lysine (K), tyrosine (Y), serine (S), threonine (T) and cysteine (C)). Accordingly, what is needed are improved methods for labeling of biological molecules for protein footprinting applications, that effectively label a higher number of amino acids with high resolution and high throughput.

### SUMMARY

Provided herein is a rapid, multiplexed radical labeling platform for protein-footprinting applications. In some embodiments, the methods provided herein use the radical precursor sodium triflinate (also referred to as sodium trifluoromethanesulfinate, sodium trifluoromethylsufinate, or CF₃SO₂Na) and tert-butyl hydroperoxide (TBHP, also referred to herein as “tBOOH”) to mediate catalyst and transition metal free hydroxyl (·OH) and trifluoromethyl (·CF3) radical production in-solution, without requiring a plasma source. Furthermore, (·CF3) and (·OH) radicals are shown herein to exhibit different reactivity, demonstrating that the approach can lead to higher resolution downstream footprinting.

In some aspects, provided herein is a plasma-free method for multiplexed labeling of a biological molecule with trifluoromethyl radicals and hydroxyl radicals, the method comprising providing a sample containing a biological molecule; and incubating the sample with a radical precursor and a peroxide for an amount of time sufficient for trifluoromethyl radicals and hydroxyl radicals to be generated from the radical precursor and interact with the biological molecule, thereby labeling the biological molecule with the trifluoromethyl radicals and hydroxyl radicals. In some embodiments, the radical precursor comprises sodium triflinate. In some embodiments, the sample comprises 100 μM to 1M sodium triflinate. In some embodiments, the sample comprises 1 mM to 100 mM sodium triflinate. For example, in some embodiments the sample comprises 50 mM sodium triflinate.

In some embodiments, the sample comprises the peroxide at a concentration of 100 μM to 1M. In some embodiments the sample comprises the peroxide at a concentration of 1 mM to 50 mM. For example, in some embodiments the sample comprises the peroxide at a concentration of 5 mM. In some embodiments, the peroxide comprises TBHP.

In some embodiments, the sample comprises a phosphate buffer. In some embodiments, the buffer comprises phosphate buffered saline (PBS) or sodium phosphate buffer (PO4). In some embodiments, the sample comprises 1 mM to 500 mM PBS or sodium phosphate buffer. In some embodiments, the sample comprises 10 mM to 100 mM PBS or sodium phosphate buffer. For example, in some embodiments the sample comprises 40 mM to 60 mM PBS or sodium phosphate buffer. In some embodiments, the sample comprises 50 mM to 5 mM PBS or sodium phosphate buffer.

In some embodiments, the sample further comprises sulfo-N-hydroxysulfosuccinimide (sNHS).

In some embodiments, the biological molecule is an isolated protein. In some embodiments, the biological molecule is a cell-membrane protein, and wherein the sample comprises live cells containing the cell-membrane protein.

In some embodiments, the sample additionally comprises a salt. In some embodiments, the sample comprises live cells containing the cell-membrane protein and a salt. In some embodiments, the salt comprises sodium chloride (NaCl), calcium chloride (CaCl₂)), potassium chloride (KCl), or sodium bicarbonate (NaHCO₃). In some embodiments, the sample comprises salt at a concentration such that the sample is isotonic with the live cells. For example, in some embodiments concentrations of sodium triflinate and the salt (e.g. NaCl) are modulated such that the sample is isotonic with the live cells present in the sample. In some embodiments, the sample comprises the salt at a concentration of 100 μM to 1M. In some embodiments, the sample comprises the salt at a concentration of 5 mM to 200 mM. In some embodiments, the sample comprises the salt at a concentration of 25 mM to 150 mM.

In some embodiments, the method comprises incubating the sample with a radical precursor and tert-butyl hydroperoxide (TBHP) for an amount of time sufficient for trifluoromethyl radicals and hydroxyl radicals to be generated from the radical precursor and interact with the biological molecule, thereby labeling the biological molecule. In some embodiments, the amount of time is 1 minute to 24 hours. In some embodiments, the amount of time is 1 minute to 120 minutes. In some embodiments, the amount of time is 30-60 minutes. In some embodiments, the amount of time is 60 minutes.

In some embodiments, the method further comprises identifying labeling of the biological molecule with the trifluoromethyl radicals and hydroxyl radicals using mass spectrometry.

### BRIEF DESCRIPTION OF THE DRAWINGS

FIG. 1A is a bar graph showing total TBHP-mediated trifluoromethyl (·CF3) labeling of BSA peptides. The percent (%) modification of peptides was calculated and averaged for two biological replicates of BSA resuspended in 50 mM PBS and labeled with 1 mM TBHP and 50 mM sodium triflinate. Samples were incubated for various times before quenching and downstream LC-MS/MS.

FIG. 1B, FIG. 1C, and FIG. 1D show optimization of buffer conditions T-BRIMB mediated CF3 labeling.

FIG. 2 shows a sequence summary of TNFα/Infliximab T-BRIMB mediated OH and CF3 radical labeling and epitope mapping results. Annotation/numbering is based on the mature TNF sequence after signal peptide removal. This sequence, after signal peptide removal, is VRSSSRTPSDKPVAHVVANPQAEGQLQWLNRRANALLANGVELRDNQLVVPSEGLYLIY SQVLFKGQGCPSTHVLLTHTISRIAVSYQTKVNLLSAIKSPCQRETPEGAEAKPWYEPIYL GGVFQLEKGDRLSAEINRPDYLDFAESGQVYFGIIAL (SEQ ID NO: 1). Primary and secondary epitopes detected by HDX are shown with a solid or dashed-line box around them respectively. Epitope regions are those that showed a significant decrease in modification after T-BRIMB in the bound vs. unbound state upon complexation with Fab compared to a non-binding partner/negative control Fab. Residues labeled with OH radicals are shown with black lettering and are highlighted in light gray and those labeled with CF3 radicals are shown in white lettering and are highlighted in dark gray.

FIGS. 3A-3C are bar graphs showing in-vitro TBHP-mediated trifluoromethyl (·CF3) and (·OH) radical protein footprinting of TNFα/Infliximab Fab epitope. The residue-level normalized fold-change in TNFα modification upon complexation with Infliximab Fab. The average, standard deviation and fold-change across conditions was calculated, then normalized to the unbound condition. Student's t-tests were performed to detect statistically significant differences in percent modification between conditions. Asterisks indicate a statistically significant change in percent modification p≤0.05 (*), p≤0.01 (**) and p≤0.0001 (***).

FIG. 4 is a graph showing TBHP-mediated hydroxyl (·OH) and trifluoromethyl (·CF3) labeling of CTLA4 peptides at two TBHP concentrations. The percent (%) modification of CTLA4 peptides was calculated for two biological replicates of CTLA4-FLAG-expressing Freestyle™ 293-F cells labeled with 1 mM or 5 mM TBHP for 30 minutes.

FIG. 5 is a graph showing T-BRIMB-mediated trifluoromethyl (·CF3) labeling of CTLA4-FLAG immunoprecipitated from FreeStyle™ 293-F cells treated with α-CTLA4 antibody Fab. The percent (%) CF3 modification of the CTLA4 MYPPPY epitope peptide was calculated for four independent biological replicates of CTLA4-FLAG-expressing FreeStyle™ 293-F cells incubated with a non-specific NIST antibody Fab control or a CTLA4-specific antibody Fab. Two-tailed two-sample equal variance (homoscedastic) Student's t-tests were performed to detect statistically significant differences in percent modification between conditions. Asterisks indicate a statistically significant change in percent modification across conditions (p≤0.05).

FIG. 6 is a graph showing TBHP-mediated trifluoromethyl (·OH) labeling of CTLA4-FLAG epitope peptide immunoprecipitated from FreeStyle™ 293-F cells treated with α-CTLA4 antibody Fab. The signal intensity of an OH-modified CTLA4 MYPPPY epitope peptide was measured for six independent biological replicates of CTLA4-FLAG-expressing FreeStyle™ 293-F cells incubated with a non-specific NIST antibody Fab control or a CTLA4-specific antibody Fab. Two-tailed two-sample equal variance (homoscedastic) Student's t-tests were performed to detect statistically significant differences in percent modification between conditions. Asterisks indicate a statistically significant change in percent modification across conditions (p≤0.001).

FIGS. 7A-7D show Coumarin-6H fluorescence assay for CF₃ radical reaction monitoring. FIG. 7A shows coumarin-6H structure and reaction conditions. FIG. 7B shows excitation and emission spectra over time to monitor the consumption of coumarin-6H (top) and the formation of coumarin-6H—CF₃ (bottom). FIG. 7C shows validation of the assay through the presence/absence of sodium triflinate and tBOOH reagents. FIG. 7D shows CF₃ peptides identified via mass spectrometry from samples quenched at different time points during a fluorescence assay to correlate coumarin-6H—CF₃ formation with the modification of peptides.

FIG. 8A shows the number of quantified CF3 peptides following T-BRIMB mediated CF3 labeling and quenching with Tris-HCl at various time points. FIG. 8B shows the percentage of CF3 labeling following T-BRIMB mediated CF3 labeling and quenching with Tris-HCl at the same time points.

FIGS. 9A-9B show sNHS acetate chemical labeling for epitope mapping. Human IL-13 was complexed in solution with the monoclonal antibody, Tralokinumab. Samples were labeled with either CF3/Ox alone, or with CF3/Ox+sulfo-N-hydroxysulfosuccinimide (sNHS). Samples were digested and compared against an IL-13+NISTmAb negative control to determine epitope residues. FIG. 9A shows the sequence of IL-13 (SEQ ID NO: 3) with crystallographic determined epitope overlaid. Residues identified in this experiment are depicted above the residue. FIG. 9B compares the structure of IL-13 with crystallographic-determined epitope highlighted (top, gray regions) and the epitope identified in this patent (bottom, gray regions).

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### 2. Plasma-Free Methods for Labeling of Biological Molecules

Radical based “protein footprinting” provides a healthy balance between throughput and resolution. Protein footprinting approaches probe changes in structure by mapping solvent-accessible surface area (SASA) via labeling of amino acid sidechains in solution with a chemical moiety, followed by quantification using MS. Applications of protein footprinting include antibody epitope localization, where regions of protein interactions are characterized by changes in SASA between two different conditions (e.g., bound and unbound to the antigen's corresponding antibody). A region showing protection, or a decreased level of labeling in the bound condition versus unbound, can therefore be an indication of a binding region.

An exemplary footprinting approach is Hydroxyl Radical Protein Footprinting (HRF), where hydroxyl (·OH) radicals are generated in solution. The OH radicals can be generated by several different methods including synchrotron radiation or laser photolysis of hydrogen peroxide (H₂O₂), otherwise known as fast-photochemical oxidation of proteins (FPOP). Recently, several of the inventors herein developed a technique for generation of radicals referred to as Plasma Induced Modification of Biomolecules (PLIMB) Minkoff, B. B., et al., (2017). Scientific Reports, 7 (1)), which utilizes an electrode with an applied potential and high energy plasma to generate microsecond-timescale bursts of hydroxyl radicals generated from water. Although HRF is a powerful technique for analysis of protein structure, motion, and interactions, it can be limited by resolution. OH radicals typically only routinely label 6 out of the 20 amino acids: methionine (M), cystine (C), tryptophan (W), phenylalanine (F), histidine (H) and tyrosine (Y). Considering these residues may only cover ˜5-30% of the protein sequence, the resolution of the data from hydroxyl radical footprinting (HRF) experiments is limited by the ability to acquire information on regions containing these six residues.

Additives have been introduced into the reaction solution during footprinting to modify amino-acid side chains with other chemical moieties, such as glycine ethyl ester (GEE) for carboxylic acids (Gau et al., 2011), dimethyl (2-hydroxy-5-nitrobenzyl) sulfonium bromide (HNSB) for tryptophan (Borotto et al., 2017) and diethyl-pyrocarbonate (DEPC). (Limpikirati et al., 2019). However, only a moderate increase in residue-level labeling coverage has been achieved. Recently, protein footprinting using trifluoromethyl radicals (·CF3) was developed (Cheng et al.). However, current protocols for footprinting using trifluoromethyl radicals generate ·CF3 radicals using a laser or synchrotron beam and photolysis of water and/or hydrogen peroxide. To date no facile, protocol exists for in vitro, multiplexed generation of OH and CF3 radicals for multiplexed labeling of proteins. Provided herein is a method for tert-butyl hydroperoxide (TBHP) mediated in vitro generation of OH and CF3 radicals from a sodium triflinate radical precursor. Notably, no transition metal catalyst, plasma, or radical synchrotron is needed for radical generation making the platform extremely amenable for routine adoption in footprinting experiments. Moreover, the methods described herein are demonstrated to be effective methods for in-vivo labeling of proteins, such as membrane-proteins, in live, intact cells.

Extensive labeling of less reactive residues remains challenging and can have negative effects on sequence coverage for applications such as epitope mapping. The methods described herein address these and other issues and provide a plasma-free method for protein footprinting with simultaneous labeling of complementary chemical moieties, including hydroxyl (·OH) and trifluoromethyl (·CF3) radicals generated in vitro with TBHP and sodium triflinate to maximize labeling coverage and increase the resolution of protein footprinting data. CF3 radicals can label all 20 amino acids, thus significantly improving labelling coverage and increasing the resolution of protein footprinting data.

Given that multiple radical species may be formed in solution with the sodium triflinate and TBHP additives, the concentration for CF3 protein footprinting applications was assessed and optimized herein. The number of radicals generated in a given footprinting experiment is a direct function of reaction conditions such as concentration of reactants, dose of the initiator and buffer composition. The resulting radical species generated by a plasma are therefore unique, and the chemical conditions for radical generation should be optimized accordingly for downstream footprinting experiments. Moreover, the resulting labeling should present a balance between maximizing the number of individual amino-acid residues labeled and population-level percent modification (i.e., average number of labels per protein molecule in a sample solution). If percent modification is too high, the protein may partially or fully denature during radical treatment, which will affect downstream data analysis and accuracy of structural characterization with the footprinting technique. The dynamic nature of multiple radicals being generated simultaneously also means that the platform should be assessed under the influence of radical scavengers, such that the system is robust and resilient against a plethora of multiple reaction conditions. Examples include but are not limited to the utilization of different buffers, pH, the presence of absence of drugs, radical scavengers, different concentrations of additives, and protein concentrations. Described herein is an optimized platform that addresses these and other concerns.

In some aspects, provided herein is a method for hydroxyl (·OH), and trifluoromethyl (·CF3) radical generation and multiplexed protein labeling. The methods provided herein generate trifluoromethyl and hydroxyl radicals without requiring plasma induction. For example, in some embodiments the methods provided herein utilize a peroxide such as TBHP to induce radical generation from the radical precursor sodium triflinate, without requiring use of a plasma source, such as a plasma needle or a plasma jet, to generate bursts of plasma. Such methods that do not utilize or rely on a plasma source are referred to herein as “plasma-free”. Plasma may be used, but is not necessary.

In some embodiments, provided herein is a plasma-free method for multiplexed labeling of a biological molecule with trifluoromethyl radicals and hydroxyl radicals. In some embodiments, the method comprises providing a sample containing a biological molecule, and incubating the sample with a radical precursor and a peroxide for an amount of time sufficient for trifluoromethyl radicals and hydroxyl radicals to be generated from the radical precursor and interact with the biological molecule, thereby labeling the biological molecule with the trifluoromethyl radicals and hydroxyl radicals. In some embodiments, the peroxide comprises tert-butyl hydroperoxide (TBHP). This method is also referred to herein as Tert-Butyl Radical Induced Modification of Biomolecules, or T-BRIMB. Although TBHP is well-suited for the methods provided herein, other peroxides may be used as an alternative to TBHP and the disclosure is not limited only to use of TBHP for plasma-free radical generation.

In some embodiments, the radical precursor comprises sodium triflinate. In some embodiments, the sample comprises 100 μM to 1M sodium triflinate. For example, in some embodiments the sample comprises 100 μM to 1M, 200 μM to 900 mM, 300 μM to 800 mM, 400 μM to 700 mM, 500 μM to 600 mM, 600 μM to 500 mM, 700 μM to 400 mM, 800 μM to 300 mM, 900 μM to 200 mM, or 1 mM to 100 mM sodium triflinate. In some embodiments, the sample comprises 1 mM to 100 mM sodium triflinate. For example, in some embodiments the sample comprises 1 mM to 100 mM, 5 mM to 95 mM, 10 mM to 90 mM, 15 mM to 85 mM, 20 mM to 80 mM, 25 mM to 75 mM, 30 mM to 70 mM, 35 mM to 65 mM, 40 mM to 60 mM, 45 mM to 55 mM, or about 50 mM sodium triflinate.

In some embodiments, the sample comprises the peroxide at a concentration of 100 μM to 1M. For example, in some embodiments the sample comprises the peroxide at a concentration of 100 μM to 1M, 200 μM to 900 mM, 300 μM to 800 mM, 400 μM to 700 mM, 500 μM to 600 mM, 600 μM to 500 mM, 700 μM to 400 mM, 800 μM to 300 mM, 900 μM to 200 mM, or 1 mM to 100 mM. In some embodiments, the sample comprises the peroxide at a concentration of 1 mM to 50 mM. For example, in some embodiments the sample comprises the peroxide at a concentration of 1 mM to −50 mM, 1 mM to 40 mM, 1 mM to 30 mM, 1 mM to 20 mM, or 1 mM to 10 mM.

A “peroxide” refers to a class of chemical compounds in which two oxygen atoms are linked together by a single covalent bond. Accordingly, peroxides have the structure R—O—O—R, where R is any element. In some embodiments, the peroxide comprises TBHP (also referred to as tBuOOH). TBHP has the chemical formula C₄H₁₀O₂. In some embodiments, the sample comprises 100 μM to 1M TBHP. For example, in some embodiments the sample comprises 100 μM to 1M, 200 μM to 900 mM, 300 μM to 800 mM, 400 μM to 700 mM, 500 μM to 600 mM, 600 μM to 500 mM, 700 μM to 400 mM, 800 μM to 300 mM, 900 μM to 200 mM, or 1 mM to 100 mM TBHP. In some embodiments, the sample comprises 1 mM to 50 mM TBHP. For example, in some embodiments the sample comprises 1 mM to −50 mM, 1 mM to 40 mM, 1 mM to 30 mM, 1 mM to 20 mM, or 1 mM to 10 mM TBHP. In some embodiments, the sample comprises 5 mM TBHP. In some embodiments, the peroxide comprises hydrogen peroxide (H₂O₂). In some embodiments, the sample comprises di-tert-butyl peroxide. In some embodiments, the sample comprises tert-butanol.

In some embodiments, the sample comprises a buffer. Exemplary buffers include, for example, phosphate buffered saline solution, tris (hydroxymethyl)aminomethane (tris), tris hydrochloric acid, ammonium bicarbonate, 4-(2-hydroxyethyl)-1-piperazineethanesulfonic acid (HEPES). 3-(N-morpholino) propanesulfonic acid (MOPS). 2-(N-morpholino) ethanesulfonic acid (MES), 2,2-Bis (hydroxymethyl)-2,2′,2″-nitrilotriethanol (bis-tris), N-(2-Acetamido)iminodiacetic acid (ADA), piperazine-N,N′-bis (2-ethanesulfonic acid) (PIPES), N-(2-Acetamido)-2-aminoethanesulfonic acid (ACES), 3-(N-morpholinyl)-2-hydroxypropanesulfonic acid sodium salt (MOPSO), 1,3-bis(tris(hydroxymethyl)methylamino)propane (bis-tris propane), N,N-Bis (2-hydroxyethyl)-2-aminoethanesulfonic acid (BES), 2-[[1,3-dihydroxy-2-(hydroxymethyl) propan-2-yl]amino]ethanesulfonic acid (TES), 3-(Bis (2-hydroxyethyl)amino)-2-hydroxypropane-1-sulfonic acid (DIPSO), 3-[[1,3-dihydroxy-2-(hydroxymethyl) propan-2-yl]amino]-2-hydroxypropane-1-sulfonic acid (TAPSO), Trizma, piperazine-1,4-bis(2-hydroxypropanesulfonic acid) dihydrate (POPSO), 3-[4-(2-Hydroxyethyl)-1-piperazinyl]propanesulfonic acid (HEPPS), N-(2-Hydroxy-1,1-bis(hydroxymethyl) ethyl) glycine (TRICINE), glycylglycine (GLY-GLY), 2-(Bis(2-hydroxyethyl) amino) acetic acid (BICINE), N-(2-hydroxyethyl) piperazine-N′-(4-butanesulfonic acid) (HEPBS), 3-[[1,3-dihydroxy-2-(hydroxymethyl) propan-2-yl]amino]propane-1-sulfonic acid (TAPS), 2-amino-2-methyl-1,3-propanediol (AMPD), N-(1,1-Dimethyl-2-hydroxyethyl)-3-amino-2-hydroxypropanesulfonic acid (AMPSO), N-cyclohexyl-2-aminoethanesulfonic acid (CHES), N-cyclohexyl-2-hydroxyl-3-aminopropanesulfonic acid (CAPSO), 1-amino-2-methyl-1-propanol (AMP), N-cyclohexyl-3-aminopropanesulfonic acid (CAPS), 4-(cyclobexylamino)-1-butanesulfonic acid (CABS), Lysogeny broth (LB) or other nutrient growth media, anything defined as a ‘biological buffer’, a biologically or physiologically-relevant salt, combinations thereof, and the like. In some embodiments, the buffer has a pH value of between 1 and 14, including but not limited to, a pH of between 3 and 9, or a pH of between 4 and 8.

In some embodiments, the sample comprises a phosphate buffer. For example, in some embodiments the buffer comprises phosphate buffered saline (PBS) or sodium phosphate buffer. In some embodiments, the sample comprises 1 mM to 500 mM PBS or sodium phosphate buffer. For example, in some embodiments the sample comprises 1 m to 500 mM, 5 mM to 400 mM, 10 mM to 300 mM, or 50 mM to 200 mM phosphate buffer. For example, in some embodiments the sample comprises 1 mM to 500 mM, 2.5 mM to 400 mM, 5.5 mM to 300 mM, 7.5 mM to 200 mM, or 10 mM to about 100 mM PBS or sodium phosphate buffer. In some embodiments, the sample comprises 10 mM-100 mM PBS or sodium phosphate buffer. For example, in some embodiments the sample comprises 10 mM to 100 mM, 20 mM to 90 mM, 30 mM to 80 mM, 40 mM to 60 mM, or 45 mM to about 55 mM PBS or sodium phosphate buffer. In some embodiments, the sample comprises 50-55 mM PBS or sodium phosphate buffer.

In some embodiments, the sample additionally comprises a salt. In some embodiments, the sample comprises live cells and a salt, wherein the salt is present at a concentration such that the sample is isotonic with the live cells. Given that sodium triflinate also impacts tonicity of the buffer, the concentrations of sodium triflinate and the salt (e.g. NaCl) can be modified to produce the desired buffer with the appropriate concentration of sodium triflinate for radical generation and the appropriate tonicity (e.g. amounts of sodium triflinate and any additional salts present in the sample) to be isotonic with the live cells in the sample. An isotonic buffer avoids unwanted hypotonic or hypertonic cell lysis. Any suitable salt that is compatible with cells and provides the appropriate buffer conditions for radical generation may be used. In some embodiments, the salt comprises sodium chloride (NaCl), calcium chloride (CaCl₂)), potassium chloride (KCl), or sodium bicarbonate (NaHCO₃). In some embodiments, the sample comprises the salt at a concentration of 100 μM to 1M. For example, in some embodiments the sample comprises 100 μM to 1M, 200 μM to 900 mM, 300 μM to 800 mM, 400 μM to 700 mM, 500 μM to 600 mM, 600 μM to 500 mM, 700 μM to 400 mM, 800 μM to 300 mM, 900 μM to 200 mM, or 1 mM to 100 mM of the salt. In some embodiments, the sample comprises the salt at a concentration of 5 mM to 200 mM. In some embodiments, the sample comprises the salt at a concentration of 25 mM to 150 mM. In some embodiments, the salt comprises sodium chloride (NaCl). In some embodiments, the sample comprises 5-200 mM NaCl. For example, in some embodiments the sample comprises 25-150 mM NaCl.

In some embodiments, the method comprises incubating the sample with the radical precursor (e.g. sodium triflinate) and the peroxide (e.g. tert-butyl hydroperoxide (TBHP)) for an amount of time sufficient for trifluoromethyl radicals and hydroxyl radicals to be generated from the radical precursor and interact with the biological molecule, thereby labeling the biological molecule with the trifluoromethyl radicals and hydroxyl radicals. In some embodiments, the amount of time is about 1 minute to 24 hours. In some embodiments, the amount of time is 1 minute to 12 hours. In some embodiments, the amount of time is 1 minute to 6 hours. In some embodiments, the amount of time is 1 minute to 3 hours. In some embodiments, the amount of time is 1 minute to 120 minutes. In some embodiments, the amount of time is between 30-60 minutes. In some embodiments, the amount of time is about 60 minutes.

In some embodiments, the method further comprises quenching the reaction after the amount of time has passed. For example, in some embodiments the method further comprises quenching the sample with a solution comprising a radical quencher. In some embodiments, the radical quencher is a sulfur-containing amino acid, such as methionine, cystine, homocysteine, or taurine. In some embodiments, the method further comprises quenching the sample with a solution comprising 10 mM to 500 mM methionine. For example, in some embodiments the method comprises quenching the sample with a solution comprising 10 mM to 500 mM methionine, 25 mM to 450 mM methionine, 50 mM to 400 mM methionine, or 75 mM to 375 mM methionine, 100 mM to 350 mM methionine, 150 mM to 300 mM methionine, or about 250 mM methionine.

In some embodiments, the sample comprises one or more radical scavengers. Radical scavengers refer to any suitable substance that removes or deactivates free radicals in the sample. Suitable radical scavengers include, for example, methionine, cystine, homocysteine, taurine, DMSO, catalase, glucose, surfactants, buffers, histidine, DTT, TCEP, lipids, sucrose, etc. In some embodiments, the sample comprises DMSO. In some embodiments, the sample comprises 2% or less DMSO.

In some embodiments, the sample comprises sulfo-N-hydroxysulfosuccinimide (sNHS). In some embodiments, sNHS is added to solution prior to the addition of TBHP. In some embodiments, sNHS is added to the solution about 30-60 minutes prior to the addition of TBHP. In some embodiments, the sample comprises sNHS at a concentration of 0.1 mM to 2 mM. In some embodiments, the sample comprises sNHS at a concentration of 0.1 mM to 2 mM, 0.2 mM to 1.9 mM, 0.3 mM to 1.8 mM, 0.4 mM to 1.7 mM, 0.5 mM to 1.6 mM, 0.6 mM to 1.5 mM, 0.7 mM to 1.4 mM, 0.7 mM to 1.3 mM, 0.8 mM to 1.2 mM, or 0.9 mM to 1.1 mM. In some embodiments, the sample comprises sNHS at a concentration of 0.5 mM to 1 mM (e.g. 0.5 mM, 0.6 mM, 0.7 mM, 0.8 mM, 0.8 mM, 1 mM). In some embodiments, sNHS enhances epitope identification compared to T-BRIMB performed in the absence of sNHS. In some embodiments, sNHS enhances epitope modification through the robust labeling of lysine, serine, and threonine residues. In some embodiments, the sample comprises 0.5 mM to 1 mM sNHS and the biological molecule is labeled (e.g. the lysine, serine, and threonine residues are labeled by sNHS) for 30-60 minutes prior to the addition of TBHP to label other residues on the biological molecule.

In some embodiments, the method labels at least 7 amino acids. In some embodiments, the method labels at least 8 amino acids. In some embodiments, the method labels at least 9 amino acids. In some embodiments, the method labels at least 10 amino acids. In some embodiments, the method labels at least 11 amino acids. In some embodiments, the method labels at least 12 amino acids. In some embodiments, the method labels valine, glycine, tryptophan, cysteine, histidine, proline, glutamine, phenylalanine, aspartic acid, glutamic acid, isoleucine, and/or tyrosine residues. In some embodiments, the sample comprises sNHS, and the method labels valine, glycine, tryptophan, cysteine, histidine, proline, glutamine, phenylalanine, aspartic acid, glutamic acid, isoleucine, and/or tyrosine residues, and additionally labels lysine, serine, and/or threonine residues.

In some aspects, the biological molecule is a nucleic acid molecule, a protein, a lipid, or a biological metabolite. In some embodiments, the biological molecule comprises a protein. In some embodiments, the sample comprises more than one biological molecule. For example, in some embodiments the sample comprises two or more different proteins of interest. In some embodiments, the protein has been digested using one or more cleavage factors. In some embodiments, the method further comprises identifying labeling of the biological molecule (e.g. evaluating multiplexed labeling of the biological molecule with the trifluoromethyl radicals, hydroxyl radicals, and nitro radicals) using mass spectrometry. In some embodiments, the biological molecule is an isolated protein. In some embodiments, a salt is particularly useful in the sample when the biological molecule is an isolated protein.

In some embodiments, the biological molecule is a cell-membrane protein. For example, in some embodiments the biological molecule is a cell-membrane protein, and the sample comprises live, intact cells containing the cell-membrane protein. Accordingly, the methods provided herein find use in labeling proteins in live cells, which is hugely advantageous for understanding of protein structure in a protein's natural setting. In some embodiments, the live cells are substantially intact. In other words, in some embodiments the cell membranes of the live cells are substantially intact and have not been ruptured or permeabilized prior to generating radicals in the sample.

The methods described herein can be utilized to determine structural information about a biological molecule, including a molecule such as a membrane protein in a live, intact cell. Biological molecules can include secondary, tertiary, and quaternary structure that precludes solvent interaction with various parts of the biological molecule. In some embodiments, the methods described herein can be used to determine whether a portion of a biological molecule is accessible to a solvent. Labeling with a radical as described herein indicates that the portion of the biological molecule is accessible, whereas the absence of labeling indicates that the portion is inaccessible. In some embodiments, the methods can be used to assess a biological molecule having solvent accessible positions and solve inaccessible positions. For example, a cleavage factor can be introduced to introduce a predictable change in structure of the biological molecules, and a comparison of radical labeling in the protein after contact with the cleavage factor can be compared to radical labeling in the protein or in an equivalent protein not contacted with the cleavage factor to provide information about accessibility.

Exemplary cleavage factors include proteases and peptidases. Nonlimiting examples of suitable cleavage factors include Trypsin (e.g., bovine), Chymotrypsin (e.g., bovine), Endoproteinase Asp-N (e.g., Pseudomonas fragi), Endoproteinase Arg-C (e.g., mouse submaxillary gland), Endoproteinase Glu-C (e.g., V8 protease) (e.g., Staphylococcus aureus), Endoproteinase Lys-C (e.g., Lysobacter enzymogenes), Pepsin (e.g., porcine), Thermolysin (e.g., Bacillus thermo-proteolyticus), Elastase (e.g., porcine), Papain (e.g., Carica papaya), Proteinase K (e.g., Tritirachium album), Subtilisin (e.g., Bacillus subtilis), Clostripain (endoproteinase-Arg-C) (e.g., Clostridium histolyticum), Exopeptidase, Carboxypeptidase A (e.g., bovine), Carboxypeptidase B (e.g., porcine), Carboxypeptidase P (e.g., Penicillium janthinellum), Carboxypeptidase Y (e.g., yeast), Cathepsin C, Acylamino-acid-releasing enzyme (e.g., porcine), Pyroglutamate aminopeptidase (e.g., bovine), and the like.

The methods described herein can be useful for a variety of applications, including quality control for biopharmaceuticals, such as determining whether a biopharmaceutical has retained its secondary, tertiary, and/or quaternary structure. The methods described herein can be used in methods of assessing a disease state in a subject, such as diseases caused by a conformational change in one or more biological molecules in a subject. For example, if a disease state is expressed by the breaking apart of a protein dimer, the methods of the present disclosure could be used to identify that the contact surfaces between the subunits of the dimer, which are normally not accessible to solvent, have become accessible to solvent. The methods described herein can also be utilized to study temperature-dependent properties of a sample of interest. For example, kinetics, protein folding, and other temperature-dependent mechanisms of interest can be studied with temperature-dependent deployment of the methods described herein. The methods described herein can be utilized to determine a rate of modification for components or sub-components within the sample of interest. For example, the methods described herein can compare the rate of oxidation (e.g. radical labeling) of two different residues on a protein of interest and can make various subsequent deductions based on the differences between those rates, such as determining a level of solvent accessibility.

In some aspects, provided herein are kits for use in the methods described herein. For example, in some aspects provided herein are kits comprising TBHP and sodium triflinate, for use in a plasma-free method of producing radicals for labeling of a biological molecule (e.g. protein). In some embodiments, the kit further comprises a buffer (e.g. PBS) and/or a salt (e.g. NaCl).

### EXAMPLES

### Example 1

This example demonstrates the development and use of a multiplexed TBHP-mediated hydroxyl (·OH) and trifluoromethyl (·CF3) protein footprinting platform for in vitro labeling of BSA.

### Optimization of In Vitro Tert-Butyl Radical Induced Modification of Biomolecules (T-BRIMB) Platform Conditions.

Varying sample conditions for the TBHP-mediated radical footprinting platform can tune the chemistry and reactivity of the resulting labeling. However, in the multiplexed platform, given the plethora of radical species in solution, their interplay and dependance on one another, and their complicated generation mechanisms and varying half-lives, it is unclear what the optimal labeling conditions will be to balance protein integrity and a high percentage of labeling that can make downstream data processing much easier. Varying reaction parameters were used herein to assess the CF3 labeling of proteins, prevent unfolding, and further understand reaction conditions of T-BRIMB-mediated multi-radical labeling in solution. Various sodium triflinate (e.g. 50 mM, 10 mM, 50 mM, 150 mM or 500 mM) concentrations and TBHP concentrations (e.g. 0.5 mM, 1 mM, 5 mM, 10 mM, 50 mM and 500 mM) in the reaction mixture were evaluated in addition to several different buffer compositions. A range of different labeling efficacies and reactivity was observed, pointing to the significance of optimization of the platform for footprinting approaches. Additionally, this platform is highly tunable and can be modified depending on the downstream application and desired level of modification. In some embodiments, footprinting conditions for T-BRIMB include protein resuspended in 55 mM PBS. 150 mM NaCl with a fixed concentration of 1 mM TBOH and sodium triflinate (50 mM). Here, incubation time in these conditions were further assessed and the labeling platform was demonstrated for the standard protein BSA.

### Optimization of Incubation Time:

Solutions of bovine serum albumin were prepared in 50 mM PBS buffer, 150 mM NaCl pH 7.4. Samples were prepared in 50 μL replicate aliquots and treated with 1 mM TBHP and 50 mM sodium triflinate additives for variable incubation times from 10-60 min. Following labeling, samples were quenched with a 5 μL solution of 250 mM methionine in PBS, pH 7.4, digested and analyzed by LC-MS/MS.

### Results:

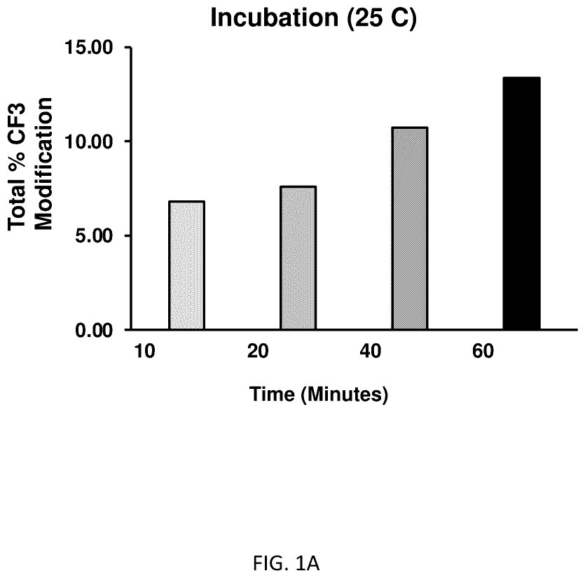

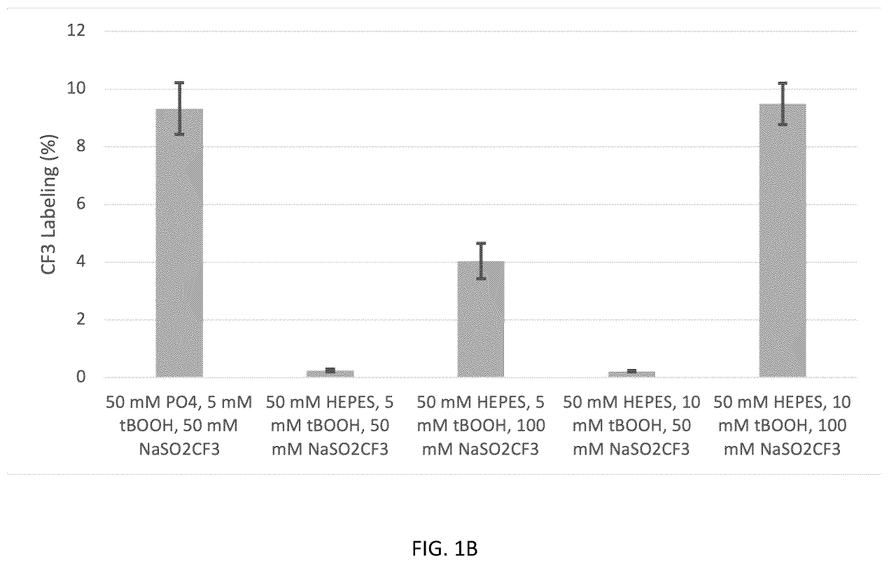

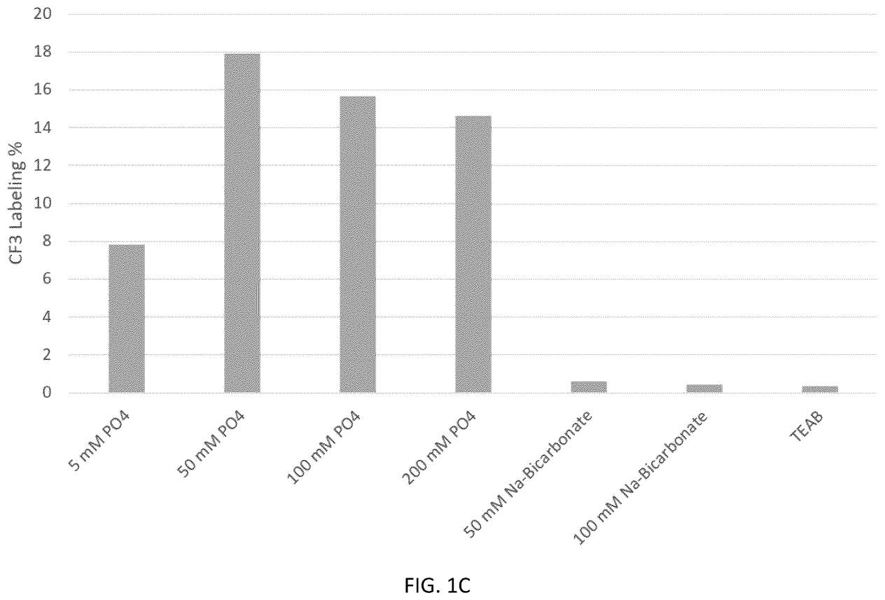

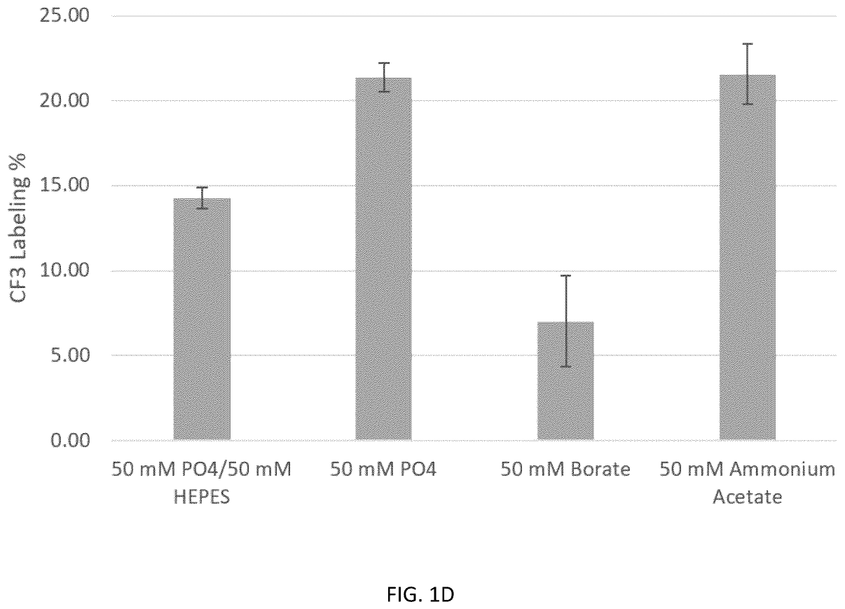

After mass spectrometry analysis, a peptide level-analysis of BSA peptides was performed across reaction conditions, which allowed for quantification and characterization of percent T-BRIMB mediated CF3 radical labeling as a function of reaction incubation time at 25 C (FIG. 1). A nearly linear increase in CF3 modification was observed with increasing incubation time from 10-60 minutes (FIG. 1). The shorter incubation times yielded a ˜5% total CF3 modification of BSA peptides, whereas the full hour led to a maximum observed CF3 labeling of ˜15% (FIG. 1). CF3 labeling was confirmed by extracting MS1 and MS2 peaks for the BSA peptide LGEYGFQNALIVR (SEQ ID NO: 2), confirming CF3 labeling on the tyrosine reside (residue 4).

### Optimization of Buffer Conditions:

Varying buffer conditions were next tested to further optimize the T-BRIMB platform. Various buffers were tested, including phosphate buffer and HEPES buffer. Specifically, the following buffers were tested:
Buffer 1: 50 mm sodium phosphase (PO4), 5 mm tBOOH, and 50 mM sodium triflinate (NaSO₂CF₃),
Buffer 2: 50 mM HEPES, 5 mM tBOOH, and 50 mM sodium triflinate,
Buffer 3: 50 mM HEPES, 5 mM tBOOH, and 100 mM NaSO₂CF₃,
Buffer 4: 50 mM HEPES, 10 mM tBOOH, and 50 mM NaSO₂CF₃.
Buffer 5: 50 mM HEPES, 10 mM tBOOH, and 100 mM NaSO₂CF₃.
All buffers contained 100 mM NaCl.

CF3 labeling was evaluated using BSA and an incubation time of 30 minutes. Results are presented in FIG. 1B. While some labeling was seen in HEPES buffer (Buffer 5), the pH of the labeling solution dropped below 5, suggesting that the buffer capacity of the HEPES buffer had been exceeded. This likely due to HEPES being a radical scavenger. As such, other buffer conditions were evaluated as follows:
Buffer 6: 5 mM sodium phosphate buffer (PO4), 5 mm tBOOH, 50 mM sodium triflinate
Buffer 7: 50 mM PO4, 5 mm tBOOH, 50 mM sodium triflinate
Buffer 8: 100 mM PO4, 5 mm tBOOH, 50 mM sodium triflinate
Buffer 9: 200 mM PO4, 5 mm tBOOH, 50 mM sodium triflinate
Buffer 10: 50 mM sodium bicarbonate, 5 mm tBOOH, 50 mM sodium triflinate
Buffer 11: 100 mM sodium bicarbonate, 5 mm tBOOH, 50 mM sodium triflinate
Buffer 12: 100 mM triethylammonium bicarbonate buffer (TEAB), 5 mm tBOOH, 50 mM sodium triflinate
All buffers contained 100 mM NaCl.

CF3 labeling was evaluated after 30 minutes. Results are presented in FIG. 1C. Data represents single replicate data (n=1). Because there was no labeling in any of the bicarbonate buffers, these buffers were not pursued any further. No changes in sample pH during labeling was observed in any of the buffers.

Additional buffers were tested.
Buffer 13: 50 mM sodium phosphate buffer (PO4), 50 mM HEPES, 5 mm tBOOH, 50 mM sodium triflinate
Buffer 14: 50 mM PO4, 50 mM HEPES, 5 mm tBOOH, 50 mM sodium triflinate (same as Buffer 1)
Buffer 15: 50 mM borate, 50 mM HEPES, 5 mm tBOOH, 50 mM sodium triflinate
Buffer 16: 50 mM ammonium acetate, 5 mm tBOOH, 50 mM sodium triflinate
All contained 100 mM NaCl.

Results are presented in FIG. 1D. Additional buffers, all contained 100 mM NaCl. The combination of sodium phosphate buffer and HEPES helped to mitigate pH changes during labeling, but lower levels of labeling were observed, likely due to the HEPES scavenging the radicals in solution. pH changes were observed in one replicate of the borate buffer and in all replicates of the ammonium acetate buffer.

Taken together, the results above demonstrate that phosphate buffers provide the best conditions for highest percentage of labeling with no detectable pH change during labeling. Specifically, phosphate buffers ranging from 5 mM to 200 mM were shown herein to have improved labeling while minimizing pH changes compared to other buffers, including HEPES, sodium bicarbonate, TEAB, borate, and ammonium acetate.

### Example 2

This example demonstrates application of in vitro Tert-Butyl Radical Induced Modification of Biomolecules (T-BRIMB) platform for epitope mapping. Mapping the binding region of an antigen to its corresponding antibody with high accuracy and resolution is of paramount importance to improve drug development success rates and decrease speed to market. To compare and test the resolution of the in vitro Tert-Butyl Radical Induced Modification of Biomolecules (T-BRIMB) platform fir mediated hydroxyl (·OH) and trifluoromethyl (·CF3) footprinting, human tumor necrosis factor alpha (TNFα) was accessed alone in solution with a non-binding control NIST Fab or in complex with the therapeutic Infliximab Fab.

### Experimental Conditions for Sample Preparation, TBHP/Sodium Triflinate Treatment, Digestion and Clean-Up:

Solutions of TNFα (Acro Biosystems) with NIST Fab or Infliximab Fab were solubilized in 5 mM PBS. pH 7.4. Antibodies were added at a 1:1 molar ratio and allowed to incubate with the antigen at 25 C for 45 minutes. After complexation, 1 mM TBHP and 50 mM sodium triflinate were spiked in and reactions were incubated at room temperature for 60 minutes for labeling. 250 mM methionine was added to quench the reaction. Chaotrope was spiked in (3M CuHCl/50 mM ammonium bicarbonate (ABC)), samples were reduced with 5 mM DTT for 10 minutes at 90° C. and alkylated with 15 mM IAA in the dark at RT for 30 minutes. Prior to digestion samples were diluted to 0.5 M GuHCl with 50 mM ABC and trypsin/LysC was added to a ratio of 1:25 (protein: enzyme) for overnight digestion at 37° C. Samples were desalted and further digested with Glu-C (in 50 mM ABC), which was added at a ratio of 1:25 (protein:enzyme) and proteins were digested further overnight at 37° C. Samples were then desalted and concentrated using C18 Stage tips (CDS Analytics).

### Experimental Conditions for LC-MS/MS:

Dried-down peptides were resuspended into 0.1% formic acid at a concentration of 100 ng/μl and injected into the LC/MS system for spectral analysis. A 30-minute chromatographic gradient from 2 to 40% acetonitrile with 0.1% formic acid was used for separation over a 2 μM, 15 cm Easy-Spray PepMap C18 column (ThermoFisher Scientific). A top-20 data-dependent acquisition was performed MS1 parameters of 120K resolving power in the Orbitrap, a scan range of 375-1200 m/z, a normalized AGC target of 300%, and MS² parameters of charge state 2-6 selection, a quadrupole isolation window of 2 Da, HCD collision energy of 30%, a normalized AGC value of 50%, and an automatic scan range starting at 110 m/z. Dynamic exclusion of 20 seconds was used after seeing an ion once. In cases where more coverage was needed a targeted or data-independent acquisition approach was utilized.

### Data Analysis for TNFα Epitope Mapping:

The ‘.raw’ MS and MS/MS data files were searched against the protein fasta sequence using Protein Metrics Byos™ for peptide spectral matching and label-free quantification, with a 1% false discovery rate (FDR) cutoff. A list of standard expected modifications and expected modifications was utilized in the database search. The following modifications were used in our search. Standard modifications: Carbamidomethyl/+57.021464 Da @ C (fixed). Variable modifications: Oxidation/+15.994915 Da @ C, F, H, M, W, Y, Dioxidation/+31.989829 Da @ C, F, M, W, Y, Cys-Oxidation/+15.994915−57.021464 Da @ C, Cys-Dioxidation/+31.989829−57.021464 Da @ C, Cys-Trioxidation/+47.984745−57.021464 Da @ C, Nitro/+44.985078 Da @ W, Y, Trifluoromethylation/+67.9874 Da @ A, C, D, E, F, G, H, I, K, L, M, N, P, Q, R, S, T, V, W, Y. The proportion of modified peptide (marked as expected variable modifications above) was calculated based on extracted ion chromatogram (XIC) relative peak areas of modified versus total peptide signal, including modified and unmodified peptides. Residue level quantification was utilized when chromatographic separation and sufficient flanking MS/MS fragments for the amino acid were present. All identifications and integrated areas were manually validated.

Differences in solvent accessibility of various regions of tumor necrosis factor-1 (TNFα) using the T-BRIMB platform were measured by accessing labeling between the two conditions. First, two solutions were prepared to map the TNFα epitope: one with TNFα and a non-binding control NIST Fab, and the other with the Infliximab Fab added at a 1:1 antibody/antigen molar ratio. Throughout this application, samples of TNFα alone will be referred to as “unbound,” and samples with antibody added will be referred to as “bound” or “complex”. For epitope mapping, a residue level-analysis was performed for identification of regions of interest showing “protection” upon complexation. Residues with a decreased level of modification upon binding with the Fab indicate “protection” or candidate epitope regions. Peptides with an increased level of modification upon complexation represent “deprotection” or areas of conformational change, which take place due to Fab binding. The peptides of interest were further validated, and residue-localized modifications were validated in Byos by verifying “Mod,” and “Delta-Mod” scores, visually inspecting MS² spectra, and adjusting extracted ion chromatogram (XIC) windows across peaks that showed consistency between samples and whose modifications could be identified with MS² hits. The residue-level analysis was utilized for peptides in the candidate epitope regions to determine which of the individually labeled amino acids show the greatest changes in solvent accessibility due to binding.

### Results:

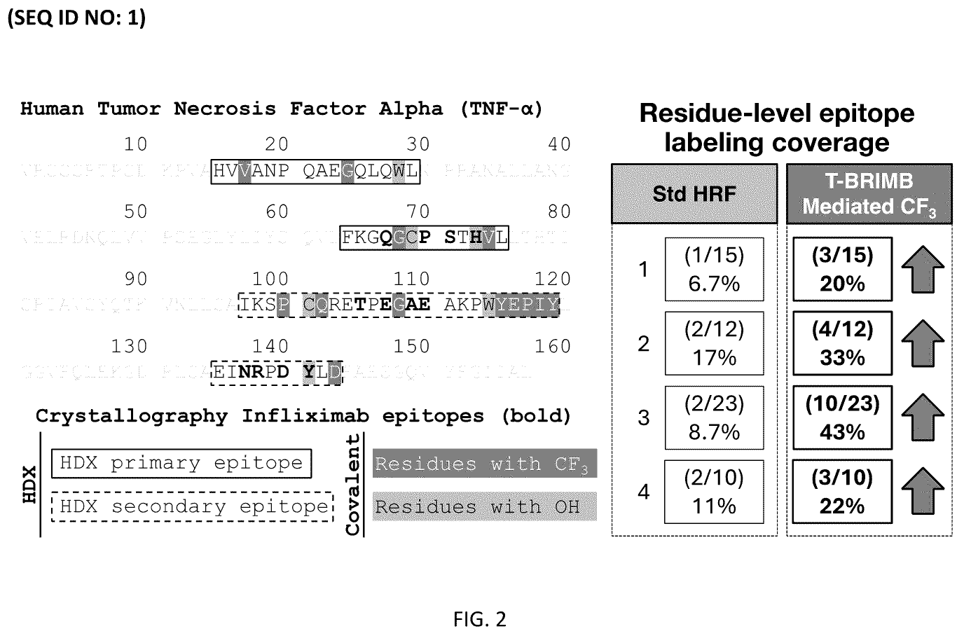

TBHP treatment in the presence of the sodium triflinate reagent, produced hydroxyl (·OH) and trifluoromethyl (·CF3) radicals in vitro to effectively label TNFα for downstream epitope mapping analysis/footprinting of the TNFα/Infliximab Fab complex using the T-BRIMB platform. First, a peptide-level analysis was performed across the entire length of the TNFα primary sequence for T-BRIMB treated samples prepared with and without the addition of the Infliximab Fab to detect differences in solvent accessibility by monitoring levels of modification upon binding/complexation. Areas of the protein showing significant protection or a decrease in OH and CF3 labeling upon complexation were deemed i.e. “epitope regions.” (FIG. 2, left panel) Residues spanning these regions correspond to the previously established crystallography data, which determined the following TNFα residues as critical for the interaction or complexation (Q57, P70, S71, H73, T105, D107, A109, D110, N137, R138, E140 and Y141) (Liang 2013). These regions were divided into and will be referred to as the “primary” and “secondary epitope regions.”

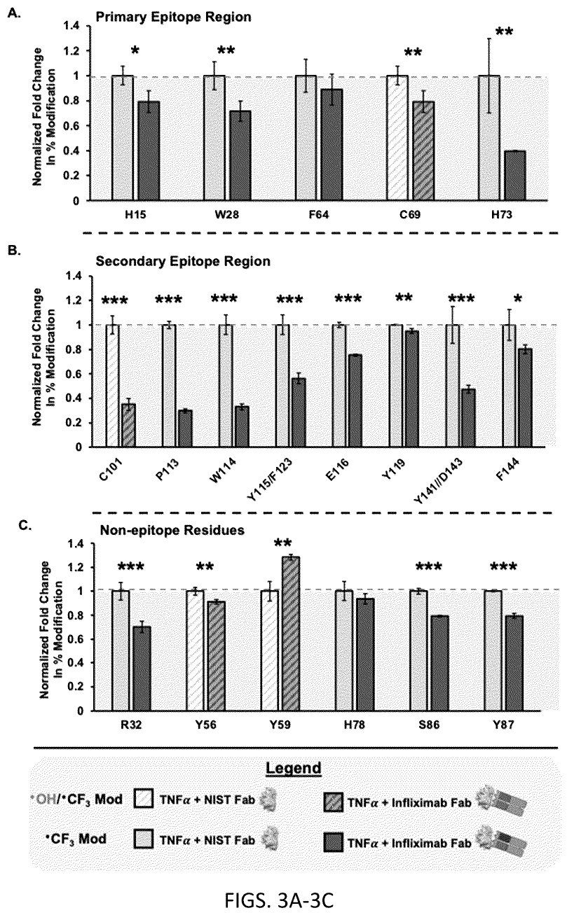

Next, the residue level labeling coverage of the TNFα antigen for standard hydroxyl radical (·OH) HRF (data collected in-house previously) vs. the T-BRIMB footprinting platform utilizing (·OH) and (·CF3) was compared (FIG. 2). Notably, while the hydroxyl radical footprinting condition labeled cysteine, tyrosine, histidine, and tryptophan residues, the (·CF3) radical labeled effectively labeled several other of the remaining 15/20 amino acids, including glutamine, and glutamic acid, residues normally “silent” to standard HRF labeling (FIG. 3). Overall, the multiplexed labeling method provided an increase in labeling coverage over each of the epitope regions of the TNFα antigen residues (FIG. 2). The platform produces both unique and complementary labeling on residues such as tyrosine for increased confidence and quantitation. For example, the multiplexed labeling allowed percent modification data to be collected with both the (·OH) and (·CF3) modification for residues such as histidine and tryptophan, increasing the confidence of data and footprinting experiment.

The fold-change in CF3 modification across residues in the epitope regions was calculated and normalized (FIG. 3). Statistically significant protection on thirteen residues spanning the epitope regions and increased the resolution of what could be obtained utilizing established methodologies was observed (FIG. 3) Residues outside of the epitope regions, such as Y57 and Y59 did not change significantly, indicating an overall preservation in protein structure during the labeling process. Overall, the resulting epitope mapped with the T-BRIMB-mediated hydroxyl (·OH) and trifluoromethyl (·CF3) methodology overlaps and is consistent with the previously published structure, indicating a high level of accuracy. The established T-BRIMB platform also both enhanced labelling coverage and significantly increased the resolution of footprinting for the application of epitope mapping.

### Example 3

This example demonstrates application of in vivo Tert-Butyl Radical Induced Modification of Biomolecules (T-BRIMB) platform for epitope mapping of membrane proteins. Membrane proteins account for a large portion of the proteome and are the targets for many drugs in development. However, mapping the HOS of membrane proteins using protein footprinting has presented a significant challenge due to the inherent insolubility and the presence of multiple transmembrane domains. Previous work has mostly focused on analyzing the soluble extracellular domain of membrane proteins. Recently, researchers have analyzed membrane proteins in vitro with HRF approaches by embedding the full-length protein in an artificial membrane such as a vesicle or lipid nano disc, however, these systems do not fully account for the diversity of components and interactors present in a in vivo cell membrane. Another study utilized a different form of covalent labeling and diethyl pyrocarbonate (DEPC) for in-vivo labeling, live-cell epitope mapping/footprinting of membrane bound tumor necrosis factor (mTNF) (Anal. Chem. 2023, 95, 18, 7178-7185). While previous covalent labeling approaches such as DEPC expanded upon the modifiable residues available with standard HRF or OH labeling, a need clearly exists for platforms yielding a wider and robust coverage of labeling for in vivo protein footprinting.

Several existing challenges include the low abundance and expression of membrane proteins. Here, a platform was developed for the expression and isolation of membrane proteins for downstream footprinting. Notably, the in vivo labeling of these proteins is also exceedingly difficult. The lipid dense cell membrane represents a significant analytical challenge when it comes to radical based covalent labeling platforms, as it can scavenge radicals and decrease the amount of protein labeling. Additionally, buffer composition to maintain pH during the oxidative labeling process, while also ensuring a proper ionic strength for cell viability, is needed. Varying sample conditions for the T-BRIMB-mediated radical footprinting platform can tune the chemistry and reactivity of the resulting labeling. Here, reaction conditions for the in vivo approach required special attention and care to further ensure maximum cell viability and labeling.

To assess the CF3 labeling of proteins, prevent unfolding, and further understand reaction conditions of the T-BRIMB multi-radical labeling in solution, various sodium triflinate (e.g. 50 mM, 10 mM, 50 mM, 150 mM or 500 mM) concentrations and TBHP (e.g. 0.5 mM, 1 mM, 5 mM, 10 mM, 50 mM and 500 mM) concentrations were tested in the reaction mixture in addition to several different buffer compositions. A range of different labeling efficacies and reactivity was observed, pointing to the importance of optimization of the platform for footprinting approaches. Additionally, this platform is highly tunable and can be modified depending on the downstream application and desired level of modification. Overall, this determined that the optimal footprinting reaction conditions for in vivo T-BRIMB included immunoprecipitated protein isolated from cells and resuspended in 50 mM PBS, with a fixed concentration of 5 mM TBOH and sodium triflinate (50 mM) (data not shown) and an incubation time of 30-60 min at 4 C.

Mapping the binding region of a membrane protein to its corresponding antibody with high accuracy and resolution is of paramount importance to improve drug development success rates and decrease speed to market. Next, to demonstrate and test the in vitro Tert-Butyl Radical Induced Modification of Biomolecules (T-BRIMB) platform, Cytotoxic T-Lymphocyte Associated Protein 4 (CTLA4) was accessed alone in live cells or after incubation and complexation in vivo with a Fab generated from a commercially available Mab.

### In-Vivo T-BRIMB Mediated Trifluoromethyl (·CF3) and (·OH) Radical Protein Footprinting of CTLA4/Fab Epitope.

### Materials and Reagents:

All chemicals and reagents were purchased from Sigma-Aldrich unless otherwise noted. Trypsin Platinum, Chymotrypsin, and 50× protease inhibitor cocktail were purchased from Promega. Optima LC-MS grade acetonitrile, water and 0.1% formic acid in water were purchased from Fisher Scientific.

### Experimental Cell Culture Conditions:

FreeStyle™ 293-F cells were cultured to mid-log phase (approximately 2.0×10⁶ cells/mL) in Gibco™ FreeStyle™ 293 Expression Medium. FreeStyle™ 293-F cells were transiently transfected with an expression plasmid containing an epitope-tagged antigen sequence with the TransIT-PRO® Transfection Reagent and incubated (37° C., 8% CO₂, 120 RPM, 48 hr). Cells were harvested via centrifugation (300×g, 3 min, RT) and washed with 50 mL PBS, pH 7.4 twice. PBS-washed transiently transfected FreeStyle™ 293-F cells were aliquoted and stored at −80° C.

### Experimental Sample Preparation and T-BRIMB Labeling:

Frozen HEK293 freestyle cells were thawed for 30 minutes on ice and resuspended in 50 mM sodium phosphate buffer, pH 7.5, 50 mM sodium triflinate, and antibody Fab (at a molar ratio of 2:1 Fab to antigen) in a total volume of 990 μL. Cells were incubated (4° C., 30 min, rotating end-over-end) to allow antibody Fabs to fully bind to the epitope-tagged target antigen. Radical labeling was initiated with the addition of 10 μL of 500 mM TBHP (5 mM final TBHP concentration) and cells were incubated (4° C., 1 hr, rotating end-over-end) to allow for maximum radical labeling. Cell viability was monitored to ensure cells were at least 90% viable before downstream protein isolation. After the radical labeling incubation, cells were centrifuged (300×g, 3 min, 4° C.) and the supernatant was discarded. Cells were quenched with the addition of PBS, pH 7.4, centrifuged (300×g, 3 min, 4° C.), and the supernatant was discarded.

### Experimental Purification and Immunoprecipitation of CTLA4:

Cells were solubilized by resuspending the cell pellet in 200 μL RIPA buffer [50 mM Tris-HCl, pH 7.8; 150 mM NaCl; 1% NP40 (v/v); 0.5% sodium deoxycholate (w/w); 0.1% SDS (w/w); 1× protease inhibitor cocktail, and 1 mM EDTA]. Cells were incubated (4° C., 1 hr, rotating end-over-end) to allow for full cell membrane solubilization and liberation of the epitope-tagged target antigen into the soluble fraction. After the solubilization incubation, membranes and insoluble proteins were separated from the soluble fraction via centrifugation (15,000×g, 10 min, 4° C.). The 200 μL supernatant was added to 6.25 μL pre-equilibrated magnetic anti-epitope affinity resin and incubated (4° C., 1 hr, rotating end-over-end) to allow for immunoprecipitation of the epitope-tagged target antigen. After the immunoprecipitation incubation, the magnetic beads were separated from the solution via a magnetic rack, and the supernatant was discarded. The magnetic beads were washed twice with 500 μL IP WASH I Buffer [10 mM sodium phosphate buffer, pH 7.5; 500 mM NaCl; 0.025% (v: v) Tween™-20; 1× protease inhibitor cocktail, and 1 mM EDTA]. The magnetic beads were washed four times with 500 μL IP WASH II Buffer [10 mM sodium phosphate buffer, pH 7.5; 500 mM NaCl]. For each wash, wash buffer was added to the magnetic beads, magnetic beads were incubated with the wash buffer (4° C., 2 min), magnetic beads were separated from the solution via a magnetic rack, and the supernatant was discarded.

### Experimental On-Bead Digestion of CTLA4:

Washed magnetic beads were then digested via addition of 50 μL 1.5 M GuHCl (in 50 mM Tris-HCl, pH 7.8; 5 mM DTT) and incubated (60° C., 10 min, 1000 rpm) to allow for the reduction and denaturation of the proteins bound to the beads. Denatured and reduced proteins were alkylated via addition of 1.5 μL of 500 mM IAA and incubated (RT, 30 min, in the dark). Alkylation reactions were quenched with the addition of 3 μL of 100 mM DTT. Proteins were digested via addition of 100 mM Tris-HCl, pH 7.8 with 1:10 trypsin platinum: protein (w: w) to a total volume of 200 μL and incubated overnight (37° C., 16 hr, 1000 rpm). Overnight trypsin digestions were further digested with 1:10 Chymotrypsin: protein (w: w) and incubated (37° C., 4 hr, 1000 rpm). Digested peptides were desalted via SPE and dried down in a SpeedVac.

### Experimental LC-MS/MS Conditions:

Dried-down peptides were resuspended into 0.1% formic acid injected into the LC/MS system for spectral analysis. The samples were analyzed using data-dependent acquisition with an Vanquish Neo coupled to an Orbitrap Elite 240 mass spectrometer (Thermo Scientific). A 30-minute chromatographic gradient from 2 to 40% acetonitrile with 0.1% formic acid was used for separation over a 2 μM, 15 cm Easy-Spray PepMap C18 column (ThermoFisher Scientific). A top-20 data-dependent acquisition was performed MS1 parameters of 120K resolving power in the Orbitrap, a scan range of 375-1200 m/z, a normalized AGC target of 300%, and MS² parameters of charge state 2-6 selection, a quadrupole isolation window of 2 Da, HCD collision energy of 30%, a normalized AGC value of 50%, and an automatic scan range starting at 110 m/z. Dynamic exclusion of 20 seconds was used after seeing an ion once. In cases where more coverage was needed a targeted or data-independent acquisition approach was utilized.

### Data Analysis for T-BRIMB-Mediated OH, CF3 Radical Protein Footprinting:

The ‘.raw’ MS and MS/MS data files were searched against the protein fasta sequence using Protein Metrics Byos™ for peptide spectral matching and label-free quantification, with a 1% false discovery rate (FDR) cutoff. A list of standard expected modifications and expected modifications was utilized in the database search. The following modifications were used in our search. Standard modifications: Carbamidomethyl/+57.021464 Da @ C (fixed). Variable modifications: Oxidation/+15.994915 Da @ C, F, H, M, W, Y, Dioxidation/+31.989829 Da @ C, F, M, W, Y, Cys-Oxidation/+15.994915−57.021464 Da @ C, Cys-Dioxidation/+31.989829−57.021464 Da @ C, Cys-Trioxidation/+47.984745−57.021464 Da @ C, Nitro/+44.985078 Da @ W, Y, Trifluoromethylation/+67.9874 Da @ A, C, D, E, F, G, H, I, K, L, M, N, P, Q, R, S, T, V, W, Y. The proportion of plasma-modified peptide (marked as expected variable modifications above) was calculated based on extracted ion chromatogram (XIC) relative peak areas of modified versus total peptide signal, including modified and unmodified peptides. Residue level quantification was utilized when chromatographic separation and sufficient flanking MS/MS fragments for the amino acid were present. All identifications and integrated areas were manually validated.

### Results:

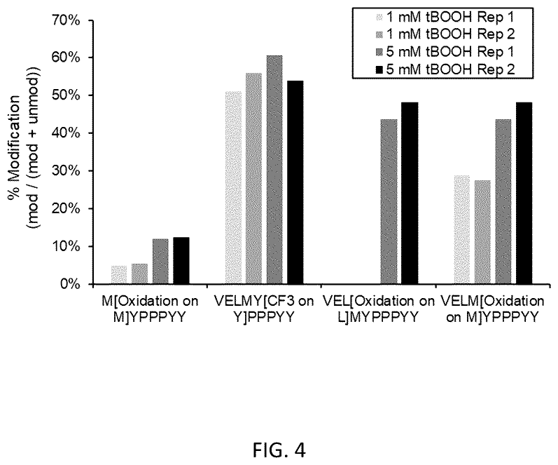

TBHP treatment in the presence of the sodium triflinate reagent produced hydroxyl (OH) and trifluoromethyl (·CF3) radicals in vivo to effectively label CTLA4 for downstream epitope mapping analysis/footprinting of the CTLA4/Fab complex using the T-BRIMB platform. 1 mM and 5 mM concentrations of TBHP were used in the presence of the sodium triflinate reagent. Results are shown in FIG. 4. TBHP produced hydroxyl (·OH) and trifluoromethyl (·CF3) radicals to effectively label CTLA4-FLAG for downstream epitope mapping analysis (FIG. 4.).

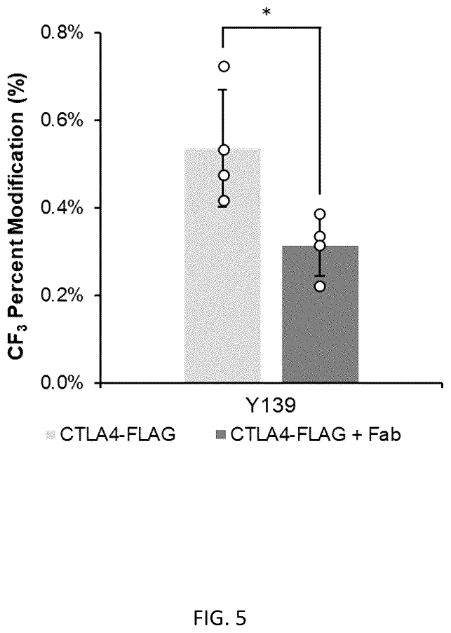

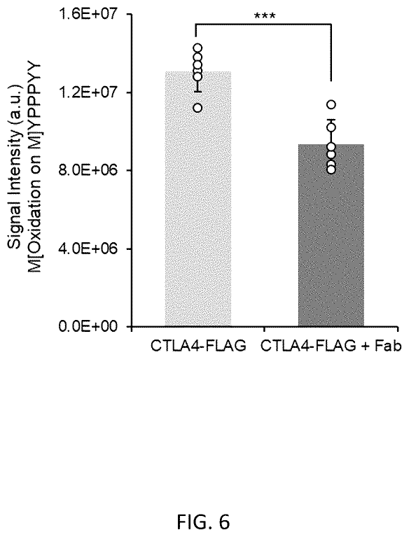

A peptide and residue level analysis was performed across the entire length of the CTLA4 primary sequence for T-BRIMB treated samples prepared with and without the addition of the commercially available experimental Fab to detect differences in solvent accessibility by monitoring levels of modification upon binding/complexation. Areas of the protein showing significant protection or a decrease in OH and CF3 labeling upon complexation were deemed i.e. “epitope regions.” Protection on residue Y135 of CTLA4 upon Fab complexation was observed, which agrees with the previously established crystallography data (Ramagopal et al., PNAS 114:21 (2017). Significant changes in CF3 labeling on the CTLA4 epitope peptide when CTLA4-expressing FreeStyle™ 293-F cells are incubated with α-CTLA4 antibody Fab were observed (FIG. 5). Significant changes in OH labeling on the CTLA4 epitope peptide when CTLA4-expressing FreeStyle™ 293-F cells are incubated with α-CTLA4 antibody Fab were also observed (FIG. 6).

In summary, the results demonstrate successful T-BRIMB mediated OH and CF3 radical labeling and footprinting of a membrane protein in the native environment, live cells.

### Example 4

Given that mass spectrometry is expensive and requires specialized instrumentation, it was next evaluated whether T-BRIMB mediated CF3 labeling can be evaluated by alternative methods. Specifically, it was investigated whether a Coumarin-6H fluorescence assay can be used to evaluate CF3 labeling.

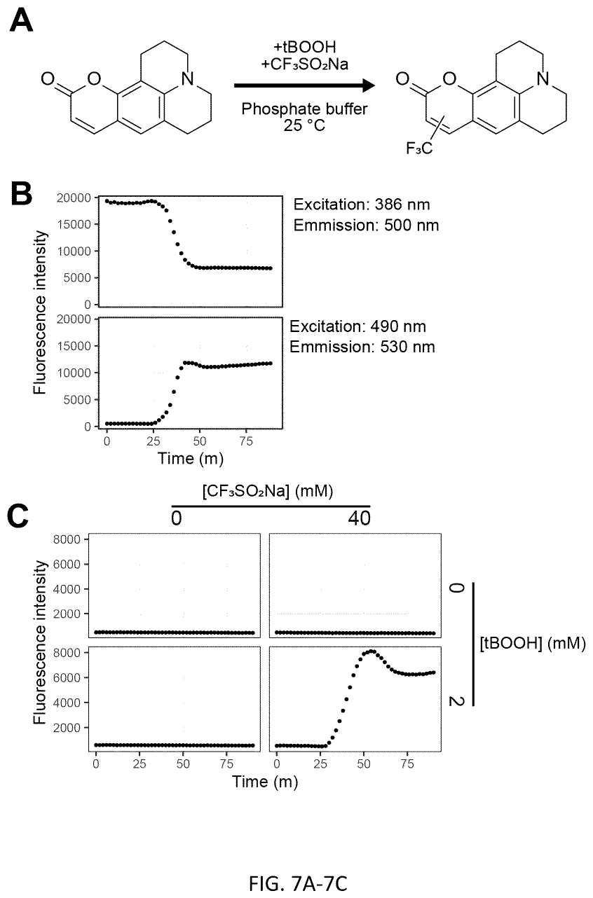

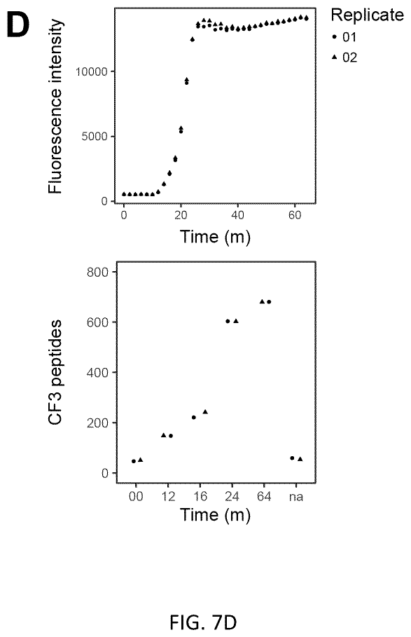

A control reaction containing sodium triflinate, protein, and tBOOH reagents was first established. Reaction mixtures contained 20 ug BSA, 2 mM tBOOH, 40 mM sodium triflinate (CF₃SO₂Na), 10 nM coumarin-6H, 50 mM phosphate buffer, and 100 mM NaCl and had a final reaction volume of 50 uL. Reactions were monitored in a plate reader every 2 minutes for 90 minutes total. Fluorescence measurements were taken every 2 minutes for 90 minutes total, first at 386 nm (ex)/500 nm (cm), then at 490 nm (ex)/530 nm (em). Results are shown in FIG. 7B.

To validate the assay, reactions were established in the presence and absence of sodium triflinate and tBOOH. Reaction mixtures contained 20 ug BSA, +/−2 mM tBOOH, +/−40 mM sodium triflinate, 10 nM coumarin-6H, 50 mM phosphate buffer, and 100 mM NaCl and had a final reaction volume of 50 μL. Reactions were monitored in a plate reader every 2 minutes for 90 minutes total. Fluorescence measurements were taken every 2 minutes for 90 minutes total, first at 386 nm (ex)/500 nm (em), then at 490 nm (ex)/530 nm (em). Results are shown in FIG. 7C.

Next, CF3 peptides were identified via mass spectrometry from samples quenched at different time points during the coumarin-6H fluorescence assay to correlate coumarin-6H—CF3 formation with the modification of peptides. Reaction mixtures contained 20 ug BSA, 2 mM tBOOH, 40 mM sodium triflinate, 10 nM coumarin-6H, 50 mM phosphate buffer, and 100 mM NaCl and had a final reaction volume of 50 uL. Reactions were monitored in a plate reader every 2 minutes for 64 minutes total. Fluorescence measurements were taken every 2 minutes for 90 minutes total, first at 386 nm (ex)/500 nm (em), then at 490 nm (ex)/530 nm (em). Samples were prepped for mass spectrometry (including a negative control containing no tBOOH, “na”). Reactions were quenched at different timepoints through the addition of 50 mM Tris buffer+50 mM methionine. Following quenching, samples were reduced with 5 mM DTT, alkylated with 15 mM IAA, and digested overnight with trypsin. Chymotrypsin was then added and allowed to digest for an additional 4 h before samples were desalted with C18 stage tips. Peptides were analyzed on an Exploris 240 mass spectrometer. Results are shown in FIG. 7D.

The results presented in FIG. 7B-7D demonstrate that coumarin-6H fluorescence is altered through the radical-mediated addition of CF3 used in the T-BRIMB platform. Moreover, the fluorescence signal can be monitored, and correlates strongly to the detection of CF3-modified peptides. This mode of analysis allows for rapid screening of buffer conditions, reagents, proteins, etc. without the need for mass spectrometry which can be expensive and time consuming, to help optimize reaction conditions on a project-specific basis.

### Example 5

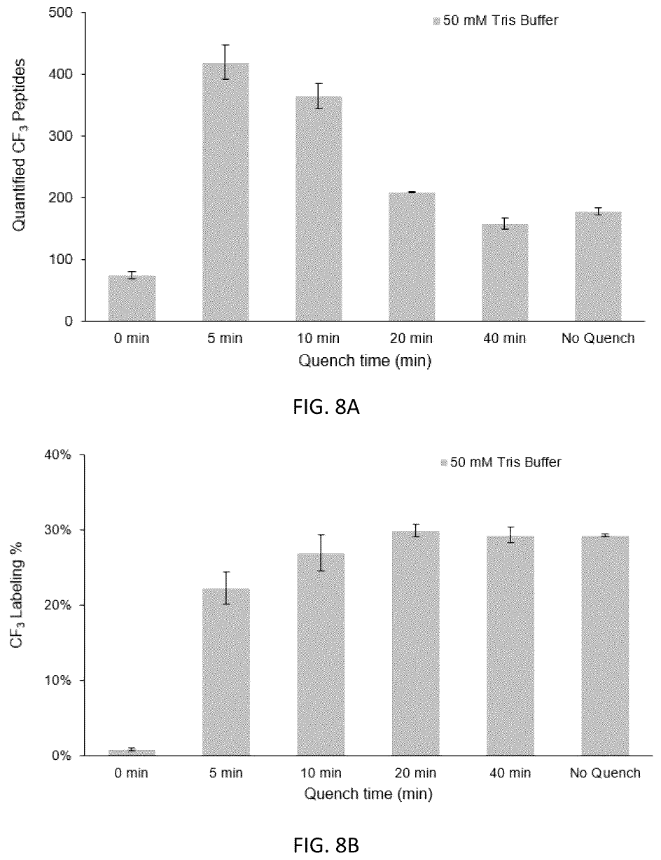

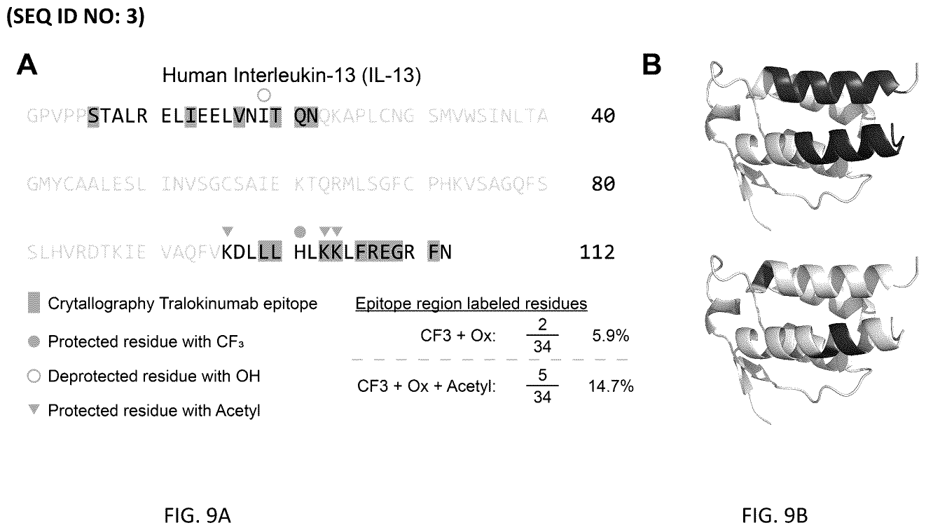

The impact of quenching the reaction with Tris-HCl was next investigated. Reaction mixtures contained 4 μg BSA, 40 mM sodium triflinate (CF₃SO₂Na), 50 mM phosphate buffer, and 100 mM NaCl and had a final reaction volume of 48 μL. BSA was CF₃ labeled with the addition of 2 μL 100 mM tBOOH (for a final tBOOH concentration of 4 mM) and incubated at 4° C., for 40 min. During the 40 minute labeling incubation, the 50 μL labeling reactions were quenched with the addition of 2.5 μL 1000 mM Tris-HCl (final concentration of 50 mM Tris-HCl) at 0 sec, 5 min, 10 min, 20 min, and 40 min timepoints. As a control, “No Quench” means no addition of Tris-HCl. Samples were prepared for mass spectrometry and CF3 labeling was analyzed. Results are presented in FIG. 8A and FIG. 8B.

### Example 6

Additional experiments were conducted to determine whether sulfo-N-hydroxysulfosuccinimide (sNHS) could enhance epitope identification compared to T-BRIMB labeling alone. 4 ug of IL-13 was complexed with either 25 ug of Tralokinumab or NISTmAb at RT for 30 minute at 4 C in a complexation buffer containing 45 uL of 50 mM phosphate, 100 mM NaCl, and 40 mM sodium triflinate. 0.5 mM sulfo-N-hydroxysulfosuccinimide (sNHS) was added to the reactions and incubated at 25 C for 45 minutes. 4 mM tBOOH was added to the reactions to initiate radical labeling, and the reactions were incubated for an additional 50 minutes. Reactions were quenched by adding 10 mM imidazole+10 mM methionine in 100 mM Tris buffer. Following quenching, samples were reduced with 5 mM DTT, alkylated with 15 mM IAA, and digested overnight with trypsin. Chymotrypsin was then added and allowed to digest for an additional 4 h before samples were desalted with C18 stage tips. Peptides were analyzed on an Exploris 240 mass spectrometer. Identified peptides were quantified and compared between samples containing Tralokinumab or NISTmAb. Modified sites displaying significant change were considered as potential epitope sites. Of the five modified residues identified within the epitope of IL-13, three of them were acetylated as part of the sNHS labeling reaction and would have otherwise gone unmodified under standard T-BRIMB labeling conditions. Inclusion of sNHS acetylation prior to T-BRIMB radical labeling significantly improved the labeling resolution and epitope identification for IL-13. This method can be readily applied to other protein footprinting applications, and may be particularly useful in cases where lysine, serine, or threonine are located within the region(s) of interest.

1. A plasma-free method for multiplexed labeling of a biological molecule with trifluoromethyl radicals and hydroxyl radicals, the method comprising:
a) providing a sample containing a biological molecule; and
b) incubating the sample with a radical precursor and a peroxide for an amount of time sufficient for trifluoromethyl radicals and hydroxyl radicals to be generated from the radical precursor and interact with the biological molecule, thereby labeling the biological molecule with the trifluoromethyl radicals and hydroxyl radicals.

2. The method of claim 1, where the sample comprises the peroxide at a concentration of 100 μM to 1 M, 1 mM to 50 mM, or about 5 mM.

3. (canceled)

4. (canceled)

5. The method of claim 1, wherein the peroxide comprises tert-butyl hydroperoxide (TBHP).

6. The method of claim 1, wherein the radical precursor comprises sodium triflinate.

7. The method of claim 6, wherein the sample comprises 100 μM to 1M sodium triflinate, 1 mM to 100 mM sodium triflinate, or about 50 mM sodium triflinate.

8. (canceled)

9. (canceled)

10. The method of claim 1, wherein the sample comprises a phosphate buffer.

11. The method of claim 10, wherein the buffer comprises phosphate buffered saline (PBS) or sodium phosphate buffer.

12. The method of claim 11, wherein the sample comprises 1 mM to 500 mM PBS or sodium phosphate buffer, 50 mM to 200 mM PBS or sodium phosphate buffer, 10 mM to 100 mM PBS or sodium phosphate buffer, or 40 mM to 60 mM PBS or sodium phosphate buffer.

13. (canceled)

14. (canceled)

15. (canceled)

16. The method of claim 1, wherein the biological molecule is an isolated protein.

17. The method of claim 1, wherein the biological molecule is a cell-membrane protein, and wherein the sample comprises live cells containing the cell-membrane protein.

18. The method of claim 17, wherein the sample additionally comprises a salt.

19. The method of claim 18, wherein the salt comprises sodium chloride (NaCl), calcium chloride (CaCl₂)), potassium chloride (KCl), or sodium bicarbonate (NaHCO₃).

20. The method of claim 19, wherein the sample comprises salt at a concentration such that the sample is isotonic with the live cells.

21. The method of claim 18, wherein the sample comprises the salt at a concentration of 100 μM to 1M, 5 mM to 200 mM, or 25 mM to 150 mM.

22. (canceled)

23. (canceled)

24. The method of claim 1, wherein the sample additionally comprises sulfo-N-hydroxysulfosuccinimide (sNHS).

25. The method of claim 24, wherein the sample comprises 0.1 mM to 2 mM sNHS or 0.5 mM to 1 mM sNHS.

26. (canceled)

27. (canceled)

28. The method of claim 271, wherein the amount of time is 1 minute to 120 minutes.

29. The method of claim 28, wherein the amount of time is 30-60 minutes.

30. (canceled)

31. The method of claim 1, further comprising identifying labeling of the biological molecule with the trifluoromethyl radicals and hydroxyl radicals using mass spectrometry.
